(19) United States
(12) Patent Application Publication
(10) Pub. No.: US 2005/0203611 A1
(43) Pub. Date: Sep. 15, 2005

(54) Synthetic vascular prosthesis

(75) Inventors: Paulus Hubertus Andreas Quax, Voorschoten (NL); Johan Hajo van Bockel, Den Haag (NL); Johanna Maria van der Bas, Leiden (NL)

Correspondence Address: SWANSON & BRATSCHUN L.L.C., 1745 SHEA CENTER DRIVE, SUITE 330, HIGHLANDS RANCH, CO, 80129, US

(73) Assignees: Nederlandse Organisatie voor toegepastnatuurwetens chappelijk, Delft (NL); Universiteit Leiden, Leiden (NL)

(21) Appl. No.: 10/494,718

(22) PCT Filed: Nov. 6, 2002
(86) PCT No.: PCT/NL02/00707

§ 371 Date: Apr. 14, 2005

(30) Foreign Application Priority Data
Nov. 6, 2001 (NL) 1019316

(51) Int. Cl. A61F 2/06

(52) U.S. Cl. 623/1.42

(57) ABSTRACT

The present invention relates to a vascular prosthesis containing an active substance that has the capacity to stimulate the ingrowth of vascular cells, into the prosthesis. The present invention further relates to a method for the treatment of a vascular prosthesis, wherein the prosthesis is provided with an active substance capable of locally inducing the ingrowth of vascular cells, into the prosthesis material. Thus, the connection between vessel and prosthesis can be improved.

This invention relates to a vascular prosthesis and to a method to treat a synthetic vascular prosthesis.

Since the beginning of reconstructive vascular surgery at the end of the fifties of the 20th century, synthetic vascular prostheses are successfully used for reconstructive surgery of the heart, aorta and the larger arteries like iliac and femoropopliteal arteries. Synthetic grafts that are being used are made of e.g. Dacron® and PTFE (Polytetrafluoroethylene), but other materials are also being used.

In U.S. Pat. No. 6,221,099 a synthetic prosthesis material is disclosed that is claimed to have improved properties over the standard prostheses made of Dacron®. The described material consists of metal wires and wires made of a second material. The second material serves as an absorbing part that can contain a drug to prevent the rejection of the graft by the body. This drug can also serve to inhibit intimal hyperplasia and can act as an anti-proliferative drug.

In order to improve prosthesis materials, a lot of attention has been paid to reducing thrombogenicity and inflammatory responses of the implanted material rather than to better healing of the prosthesis.

For example, JP 05 076588 A describes a vessel comprising a tube wall, which tube wall comprises a porous polymer compound. At the inner or outer surface of the tube wall, a substance stimulating and inducing a blood vessel endothelium cell is provided. The vessel is reported to have antithrombotic capability, apparently by the formation of a layer of endothelium at a surface of the vessel. It has been found that although lateral growth of endothelium has antithrombotic capability, such layer does not significantly contribute to the strength of the anastomotic site.

WO 00/30698 relates to a tissue/implant interface comprising an implant and a bioactive polymer layer adjacent to the outer surface of the implant. The implant interface is primarily designed to avoid uncontrolled inflammatory response and tissue fibrosis, at the surface of implanted devices, in particular biosensors. In order to achieve this, polymer layer contains one or more tissue response modifiers, such as anti-inflammatory agents, antifibrotic agents and/or anti-proliferative agents.

U.S. Pat. No. 5,866,113 focuses on enhancing infection resistance of a medical device, such as a device made of biodegradable polyurethane. A biodegradable material is not considered to be particularly suitable for durable vascular prostheses for replacement of or in larger vessels in humans, such as grafts or heart valves for long-term implantation, since such implants would likely deteriorate and potentially put the patient's life at risk. The medical device is provided with a surface graft matrix. Biomolecules are covalently coupled to the matrix, mainly at the outer portion. In the biomolecules, pharmaceutical agents, such as anti-inflammatory and antithrombotic agents, may be located.

U.S. Pat. No. 5,800,541 describes a method to prepare a collagen-hydrophilic synthetic polymer composition. The composition is reported to have a low immunogenicity and is suggested to be suitable for use in a variety of medical applications such as in drug delivery systems or in the manufacture of a formed implant. The material can be provided with a bioactive agent, e.g. an antithrombotic agent.

The colliding document WO 01/82992 relates to an alternative to synthetic implants. According to this publication synthetic DACRON and PTFE vascular prostheses are not ideal in arterial reconstructions, due to high risk of thrombosis and anastomotic hyperplasia. Rather than trying to reduce these risks by modifying the surface of the synthetic prosthesis (as has been proposed before), a different approach is described. As a basic structure for a vessel-prosthesis a decellularised vascular tissue (a biological material) is used instead of a synthetic material. An anti-thrombogenic compound and a growth factor are linked to the vascular tissue. Potential drawbacks of this approach include availability (if donors of the same species should provide the material) and ethic considerations, in particular if vascular tissue of a different species is used (xenotransplantation).

Besides relevant factors discussed above (such as immunogenicity, and thrombogenicity), it is also of paramount importance that (synthetic) prostheses, especially vascular prostheses, are well incorporated into the human body. This holds in particular for the anastomosis between an artery and a vascular prosthesis. Unfortunately, as a rule, healing is incomplete and the strength of the anastomotic site remains largely dependent on the suture line (even after many years). If the suture breaks, anastomotic dehiscence can occur and a “false” aneurysm can be formed, which may result in a fatal rupture of the anastomosis. The inventors contemplated that, if healing could be induced, the formation of complications like false aneurysm formation could be avoided.

However, several problems are associated with the use of synthetic grafts that replace diseased arteries. First, it is well known that knitted/woven prostheses dilate during the first two years after implantation. Nunn et al demonstrated, among others that the maximum percent dilation for any part of the grafts ranged from 26% to 367% with a mean of 94% (Nunn D B, Carter M M, Donohue M T, Hudgins P C., Postoperative dilation of knitted Dacron aortic bifurcation graft. J Vasc Surg (1990) 12; 291-297). Some of their patients had generalized and saccular dilation of the aortic portion of the prostheses and an anastomotic aortic aneurysm.

Secondly, it is well known that “true” and “false” aneurysms may occur during long term follow-up. In previous reports, evaluating long term patient material, it has been found that a high prevalence of false aneurysms is abundant.

False (or anastomotic) aneurysm formation during follow-up is usually associated with considerable morbidity and mortality. False aneurysms may rupture just like normal aneurysms and may cause severe bleeding shock and death of the patient if the aneurysm is located in the abdomen or thorax.

Healing of a vascular prosthesis to the vessel wall of arteries, such as the aorta, is also very important in endovascular aneurysm repair (EVAR), which is an alternative to open conventional repair, in which the aneurysm is replaced by a synthetic vascular prosthesis. With EVAR, a rupture of e.g. an aortic or iliac aneurysm can be prevented. In this type of repair, for example an aortic prosthesis-combined with stents loaded in a sheath is introduced via the femoral artery in the aorta guided by fluoroscopy. Subsequently, the prosthesis is deployed at the proper location with the stents at the proximal and distal “landing zones” fixing the graft to the aortic wall. As a result the aneurysm is excluded from the arterial circulation.

A serious problem with EVAR is endoleak. An “endoleak” is defined as flow within the aneurysmal sac between the stent graft and the aortic wall. Endoleakage may be caused by insufficient sealing or by migration which is caused by insufficient attachment of the graft to the aortic wall. Again, healing of the graft to the arterial wall is incomplete and the quality of the connection between the prosthesis and the arterial wall remains primarily dependent on the stent. Insufficient fixation may cause migration of the prosthesis and may result in endoleakage. Endoleakage causes blood pressure in the aneurysmal sac and may ultimately result in rupture of the aneurysm. A serious problem is that the presence of endoleakage may be very difficult to detect with conventional imaging (ultrasound, duplex, computer tomography, MRI or angiography). As a result, aneurysms effectively treated with EVAR, from which the clinician thought that it had been performed successfully and without endoleakage, have been ruptured causing death of patients.

Another problem is migration of the endoprosthesis, the implanted prosthesis, after EVAR. Again, due to incomplete and insufficient healing the continuous pulsatile blood stream also may cause the endograft to migrate distally with time. This may result in endoleakage and exposes the patient to the risk of rupture, bleeding and death.

It is an object of the present invention to provide an improved synthetic prosthesis, in particular an improved synthetic vascular prosthesis.

Another object is to reduce any of the above mentioned problems. Any additional purposes of the present invention will be elucidated in the description below.

It has been found that an improved synthetic prosthesis, such as a vascular prosthesis, can be provided by applying to a material forming the prosthesis (such as a fabric forming the prosthesis) a substance that can stimulate the ingrowth of cells, such as vascular cells into the prosthesis material.

Therefore, the present invention relates to a prosthesis, such as a vascular prosthesis, containing a substance capable of inducing or stimulating the ingrowth of cells, such as vascular cells, into the prosthesis material.

In particular, the present invention relates to a prosthesis wherein said substance is present in, on or at a material forming the basic structure of the prosthesis, wherein the basic structure contains voids, such as pores, to hold vascular cells. The voids allow ingrowth and/or adhesion of the cells after implantation of the prosthesis into a patient. More in particular the invention relates to a vascular prosthesis wherein said substance is present in, on or at said basic structure forming the tubular wall of a synthetic vascular graft or in, on or at a material forming a synthetic heart valve or a part thereof, such as a part that is to be contacted with the heart tissue.

Preferably the active substance is at least provided in at least part of the voids of the material.

For a substance capable of inducing or stimulating the ingrowth of cells, in particular for a substance capable of inducing or stimulating the ingrowth of vascular cells, into the prosthesis material, the term “active substance” will be used within this application.

A vascular prosthesis as used herein is a prosthesis that can be used in the heart, in any other blood vessel or in any other vessel, duct or canal in the animal or human body. Examples for vascular prostheses in line of the present invention are vascular grafts, such as cardiovascular grafts, heart valves, stents, drains, bile ducts etc.

The term prosthesis material as used herein is meant to describe the material that forms the basic structure of the prosthesis, e.g. a fabric forming the tubular structure of a vascular graft or a material forming the structure of an artificial heart valve.

The basic structure of the prosthesis comprises voids. The term voids should be interpreted broadly and in particular includes any spaces in the basic structure that allow migration of vascular cells into the structure.

The voids should have suitable dimensions to allow migration of the cells of the vessel wall or a precursor thereof into the void. The diameter is typically more than the diameter of the cell and the depth of the voids should be deep enough to allow the formation of a cellular structure of a sufficient thickness to bond with the adjacent tissue. The skilled person will know how to choose suitable dimensions. The voids are preferably such that the ingrowth is possible essentially throughout the basic structure of the prosthesis, e,g. such as is possible with a prosthesis containing a basic structure having open pores. It is possible though to provide a prosthesis wherein the ingrowth is not throughout the thickness of the basic structure, as long as the ingrowth is deep enough to allow an improved connection between the ingrown cells in the prosthesis and the adjacent tissue.

It is for example possible to provide a basic structure which comprises filaments, e.g. of a biocompatible and biostable material such as a material that is suitable to form the basic structure, at or on one or more surface regions of the basic structure. The spaces between the filaments may serve to hold the vascular cells after implantations. The skilled person will know how to choose suitable materials and design for the filaments, e.g. from those commercially available and/or described in the related literature. Such materials with filaments, also known as “velours” materials are for example commercially available from Meadox/Boston Scientific, Vascutek/Sulzer, Bard or Braun. Very suitable for example for a basic structure in a graft prosthesis is a double-velours type material, i.e. wherein both inner- and outer surface are provided with filaments.

Voids may be formed by slits or indentations at at least one surface of the basic structure.

Preferably the voids have the form of pores. The term pores should be interpreted broadly. For example, the interstitial space in a basic structure constructed of fibres (e.g. a woven or knitted material) may serve as pores, the pores may have the form of holes in the basic structure or the porous basic structure may be a spongy material.

Preferably, at least a substantial part of the pores are open pores, preferably at least the majority of the total pore volume is provided by open pores. Open pores extend from one surface of the prosthesis to another, e.g. from the inner wall to the outer wall of a arterial/venous graft.

Preferably at least part of the voids, such as pores, are filled with one or more active substances, biodegradable compounds and/or components that degrade or are released (e.g. dissolved), under physiological conditions. Thus, at least some time after implantation of the prosthesis, voids become accessible to one or more vascular cells or precursors thereof.

It has been found that after implantation of a prosthesis according to the invention, effective ingrowth and/or adhesion of cells into the basic structure forming the prosthesis is accomplished. As a result, effective healing occurs at the anastomosis of an implanted prosthesis according the invention. This results in a good incorporation of the synthetic prosthesis in the body and the blood vessel in which the anastomosis is made. A prosthesis according the invention can form a firm connection (“biological healing”) to the adjacent tissue (in particular the vessel wall) in which the prosthesis is implanted. This results in complete healing of the prosthesis to the tissue (such as the vessel wall), such that the attachment of prosthesis to the tissue is not dependent anymore of a suture line (conventional repair) or stent (endovascular repair). This prevents both rupture of a false aneurysm and endoleakage e.g. due to distal migration of the vein graft.

A synthetic heart valve according the present invention, may impart a faster and more complete healing of the valve, resulting a in better attachment to the vessel wall and a reduced risk for leakage and thus reduces the need for re-interventions to correct the problems that occur due to incomplete healing of a synthetic heart valve.

Preferably, the prosthesis contains an active substance that induces and/or stimulates intimal hyperplasia and neointima formation c.q. induction and/or stimulation of smooth muscle cell proliferation and migration, preferably in a part of the tissue that is in direct contact with the basic structure containing the active substance. The formed neointima can grow invasively in and through the prosthesis material in such a way that a firm attachment can be obtained of a intravascularly-placed endovascular prosthesis to the vessel wall.

In a preferred embodiment, an active substance in a prosthesis according the present invention induces and/or stimulates the formation of tissue that enhances the fixation. In a particular preferred embodiment an active substance is present that is capable of stimulating fibrosis, i.e. the accumulation of fibrocellular tissue by the tissue, e.g. vessel wall, at and in the prosthesis material. This may result in an extra firm attachment of the prosthesis material and the vessel wall.

It is especially remarkable that a graft prosthesis according the present invention, containing an active substance that induces and/or stimulates intimal hyperplasia and/or smooth muscle cell proliferation, has improved characteristics. After all, the experience in clinical practice would suggest the incorporation of active substances into the graft prosthesis that have opposite effects, i.e. the prevention of intimal hyperplasia and neointima formation.

Preferably the active substance is a growth factor. Examples of suitable growth factors are growth factors of the fibroblast growth factor family, e.g. bFGF or aFGF, of the platelet-derived growth factor family (PDGFs) and of the transforming growth factor family, e.g. TGFβ.

Very good results have been obtained using a prosthesis containing fibroblast growth factor (FGF). Of the fibroblast growth factor family, bFGF is a highly preferred growth factor to be used, inter alia because of its good capacity to bind to a coating of prosthesis material. Of course it is possible to apply a combination of active substances to a prosthesis according the present invention.

As a basic structure of the prosthesis (the untreated prosthesis) any kind of synthetic material can be used, as long as it is sufficiently biocompatible and biostable. A material is considered to be biostable, if it does not substantially degrade (chemically, by resorption or otherwise) under physiological conditions, for a prolonged time, typically for the life-time of the patient. More in particular, the material should preferably not substantially degrade under physiological conditions during a period of at least 25 years, more preferably of at least 30 years.

The term synthetic material is used herein to distinguish the material from biological tissues (including modified biological tissues)—such as heart valves, blood vessels and the like—and biological tissue materials, such as collagen.

For instance a commercially available prosthesis can be used, such as a graft made of Dacron® or another condensation polymer obtainable from ethylene glycol and terephthalic acid, or of polytetrafluoroethylene (PTFE), such as Teflon®. Other suitable materials are e.g. polyamide (e.g. nylon), polyester, metal (e.g. nitinol, steel, etc.), polyurethane, polycarbonate-urethane, silicon, polyolefin, Orlon, Vinyon-D or any combination of these materials.

Dacron® and Teflon® or any comparable condensation polymer obtainable from ethylene glycol and terephthalic acid respectively PTFE, are particularly preferred. These materials are inexpensive, easy to handle, have good physical characteristics and are in general thought to be suitable for clinical application. Furthermore, no special precautions are required to prevent corrosion as may be the case when metals are used. A prosthesis, in particular a graft, comprising a basic structure of any of these two types of polymers has been found to be particularly advantageous, when it is provided with an impregnation comprising a biopolymer, such as a collagen and/or a heparin, which impregnation further comprises a growth factor, in particular an FGF.

The basic structure of the prosthesis preferably has a certain degree of openness (in the case of a porous basic structure also referred to as porosity), as is the case with a spongy, a woven or a knitted graft material. The desired degree of openness and the mean void diameter (in the case of pores: pore-size) depend on the application. For grafts for instance, the mean void diameter, more in particular the mean pore-size, may be 5-150 μm. Preferably the mean-pore size is in the range of 10 to 150 μm, more preferably in the range of 30 to 100 μm, even more preferably from 55 to 65 μm.

With respect to void-size distribution, it has been found that preferably at least the majority of the voids (such as pores), more in particular 80-100% of the voids (such as pores), has a diameter in the range of 5-150 μm, more preferably a diameter in the range of 10 to 100 μm, even more preferably a diameter in the range of 30 to 65 μm.

Obviously, at the time of implantation, the voids—e.g. pores—may be filled with active substance and/or an impregnation, which gradually dissolves or degrades after implantation to allow ingrowth of vascular cells

To the vascular prosthesis, a coating, lining or impregnation can be applied. The term coating is used herein to describe a layer that is at least provided at one or more surfaces of the prosthesis. The term lining is used herein to describe a layer that is provided at the inner surface of a prosthesis. The term impregnation is used herein to describe the presence of on or more components inside the basic structure, in particular in the voids—such as pores—of the basic structure. The impregnation may partially or fully fill the voids. Preferably the impregnation is provided as a layer at least partially covering the inner surface of the voids, whilst maintaining a sufficient openness (porosity) to allow migration of endothelial cells or precursors thereof in the voids.

The coating, lining and/or impregnation may be provided to reduce the openness (porosity) of the graft at the time of implantation, e.g. avoid or at least to reduce blood loss through a porous graft. Such a coating/lining/impregnation can also contribute to the biocompatibility of the prosthesis material and/or the realization of a certain release pattern of one or more active substances.

Further, it may serve as a carrier for the active substance(s). The active substance(s) can be blended with the coating/lining/impregnation material or it can be attached to the surfaced thereof. Active substance(s) may be bound physically, by ion-ion interaction or covalently. For practical reasons it is preferred to bind active substance(s) non-covalently. In addition it has been found that thus a prosthesis with a favourable release-pattern for the active substance(s) is obtained.

Good results have been obtained with at least partially biodegradable coatings, linings or impregnations.

Particularly good results have been achieved with a porous prosthesis provided with an impregnation, present in at least part of the pores. The active substance may form part of the impregnation and/or be provided at the inner surface thereof.

Any suitable compound or composition can be used for the coating, lining or impregnation of the prosthesis material. Very suitable are, amongst others, collagens, heparins, gelatines, fibrin, polyactides, polyglycolides, polygluconates, polydioxanons, elastins, glycosaminoglycans, fibronectins, laminins, proteoglycans, albumin and globulins, or any combination of two or more of the compounds. Of these compounds collagen in combination with heparin are highly preferred. A very suitable collagen is bovine collagen or the like, e.g. collagen derivable from bovine achilles tendon.

Collagen has been found to be very suitable as a carrier material for the active substance(s). For practical reasons the collagen may be cross-linked. By altering the degree of cross-linking, the skilled person will be able to influence the degradation rate of the carrier material and the release pattern of the active substance. The skilled person will know how to cross-link the material in a suitable way to obtain a desired release pattern and degradation rate based upon the information disclosed herein and common general knowledge. Particular good results have been achieved with a prosthesis impregnated with a collagen and with active substance, such as FGF, in particular bFGF.

The present invention provides in a prosthesis from which the active substance may be released in a controlled manner, under physiological conditions. This can be achieved for instance by using a suitable coating or impregnation that is biodegradable.

Very suitable for obtaining a prosthesis with a constant release of an active substance, such as bFGF, during a certain time period is a prosthesis provided with collagen, heparin and the active substance.

Furthermore, the present invention relates to a method for the production/preparation of a vascular prosthesis, in which the prosthesis contains an active substance capable of inducing and/or stimulating the local ingrowth of vascular cells into the prosthesis.

The active substance can for instance be applied to the prosthesis in a pure form or in any other composition such a (diluted) solution, emulsion or dispersion. The active substance can, for instance, be applied very suitably to the prosthesis by means of impregnating or coating the active substance, or any composition containing the active substance, to the prosthesis material.

Further, it is possible first to apply a coating, lining or impregnation to the prosthesis and then add the active substance to it, or just the other way around, first apply the active substance and then apply the coating, lining or impregnation to the prosthesis material.

In a much preferred embodiment, first a biocompatible material, preferably collagen, is applied to the prosthesis material, to which in a next step heparin or a heparin-like substance is added (which may enable and/or enhance subsequent binding of an active substance), after which an active substance, preferably a growth factor, such as basic fibroblast growth factor, is applied.

The present invention further relates to the use of a fibroblast growth factor, preferably bFGF or FGF2, in the manufacture of a medicament for the stimulation of the ingrowth of vascular cells into an endovascular prosthesis. In a preferred embodiment the fibroblast growth factor, e.g. bFGF or FGF2, is used in the manufacture of a medicament for the stimulation of intimal hyperplasia and smooth muscle cell proliferation and migration into a vascular prosthesis.

In a much preferred form of the medicament the prosthesis acts as an application device, or as an administration unit or dosage unit, (of the active substance of) the medicament.

The present invention relates to a composition for use in endovascular reparation (EVAR), in which said composition comprises one or more stents in a graft according the present invention and wherein the graft is surrounded by a sheath, e.g. a tube like cover.

A graft in such a composition comprises a hollow tubular structure (i.e. at least in expanded state it is hollow), in which the inner wall forms a circumference of a central passage way in which the passage way extends along the longitudinal direction from one end of the tubular structure towards the other end of the structure; one or more stents are placed in the central passage way of the graft, the stent or stents and the graft are expandable in radial direction; the outer wall of the graft in the preferred composition is enveloped with a cylindrical sheath, wherein the composition has a diameter and length suitable for catheter-based delivery to a vascular segment, e.g. an arterial segment, to be treated. Herein the catheter may be positioned via entry in e.g. the femoral artery (or any other possible entry, such as e.g. the aorta or the illiac artery). Such a composition has been found particularly suitable for treating an aneurysm in the aorta.

Once a said composition is placed at the appropriate site in the blood vessel, the sheath-surrounding the graft can be removed, e.g. according the procedures known from the standard EVAR procedure currently in use. The graft containing the stent, or stents, can then expand in radial direction in such a way that the outer wall of the graft is positioned in direct contact with the vessel wall. Subsequently, under influence of the active substance in the graft that can induce and/or stimulate ingrowth of vascular cells, as described herein, the graft may from an firm connection with the vessel wall. The said sheath and stent(s) may be of any material suitable, preferably, but in no way limited to, those currently in use for the standard EVAR procedure.

The present invention further relates to a novel method for the treatment of a (cardio)vascular disorder, which method comprises the application of an endovascular prosthesis according the present invention to the vascular system of a patient.

In a preferred embodiment of treating a (cardio)vascular prosthesis according to the invention or a composition according to the invention, comprising a graft, a stent and a sheat, is placed into the vacular system by a catheter-delivery based minimal invasive endovascular technique. In particular, the present invention relates to a method to induce and/or stimulate the healing of an endovascular prosthesis to the vessel wall after implantation of the prosthesis.

Very good results have been obtained using a method in which the endovascular graft prosthesis is placed into an artery to repair an aneurysm. In that way it has been found that that rupture of an aneurysm, e.g. in the aorta or any other artery, can be prevented. Further, it has been found that after such a treatment only very little or no endoleakage occurred between the graft and the vessel wall. Also the occurrence of distal migration of the implanted graft can be strongly reduced or or even prevented totally.

A prosthesis according the present invention is very suitable for application as an endovascular graft in the treatment of abdominal or thoracic aorta aneurysms (or any other kind of aneurysm), but can be applied to material used for other endovascular grafting procedures, such as percutaneous in situ coronary venous arterialization (PICVA) as described by Oesterle et al. (Oesterle S N, Reifart N, Hauptmann E, Hayase M, Yeung A C: Percutaneous in situ coronary venous arterialization: Report of the first human catheter-based coronary artery bypass. Circulation 2001;103:2539-2543). In this procedure a catheter based coronary artery bypass is performed based on the construction of a fistula from the proximal coronary artery to the coronary vein using an endovascular graft that requires healing to the vessel wall to prevent endoleakage.

Further, the present invention can be applied on many other devices that are placed in a intra(cardio)vascular setting and suffer from problems that are related to the lack of healing to the vessel wall. This may be a device such as a heart valve.

The present application will now be illustrated while referring to the following examples. It is to be noted that these examples merely serve to illustrate the invention, not to restrict it.

### EXAMPLE 1

Induction of Neointima Formation/Intimal Hyperplasia in Organ Cultures of Human and Pig Aorta

In contrast to saphenous vein organ cultures (Slomp J. Gittenberger-deGroot A C. van Munsteren J C. Huysmans H A. van Bockel J H. van Hinsbergh V W. Poelmann R E. Nature and origin of the neointima in whole vessel wall organ culture of the human saphenous vein. Virchows Arch 1995; 428:59-67), cultures of aortic wall segments, neither human nor porcine, do not spontaneously develop a neointima in culture medium containing 30% Fetal Calf Serum. However the formation of a neointima can be induced by addition of growth factors to the culture medium, as disclosed in the following described experimental example.

Segments of porcine aorta were collected in sterile RPMI (Roswell Park Memorial Institute) 1640 culture medium supplemented with 20 mmol/l HEPES buffer, 4 IU/ml sodium heparin, 2.5 μg/ml, 100 U/ml penicillin, 100 μg/ml streptomycin, 2 mmol/L L-glutamine and 2.5 μg/ml amphotericin-B, and transported to the laboratory on ice. After removing the adventitia and fat tissue, the aorta segment was opened along its longitudinal axis. A small piece was cut of for analysis by histochemistry as t=0 reference sample. The remaining part of the aorta was fixed by needles in a petri-dish coated with Sylgard 184 silicone elastomer (Dow Corning), with its endothehal surface upward. The aorta segments were cultured for a period of 4-5 weeks at 37° C. in humidified atmosphere of 5% CO₂ in culture medium (sterile RPMI 1640 supplemented with 20 mmol/l HEPES buffer, 2.5 μg/ml, 100 U/ml penicillin, 100 μg/ml streptomycin, 2 mmol/l L-glutamine and 2.5 μg/ml amphotericin-B) supplemented with 30% heat-inactivated fetal calf serum. Furthermore, basic Fibroblast Growth Factor (bFGF 10 ng/ml) was added every time the culture medium was replaced. The medium was replaced every 2-3 days.

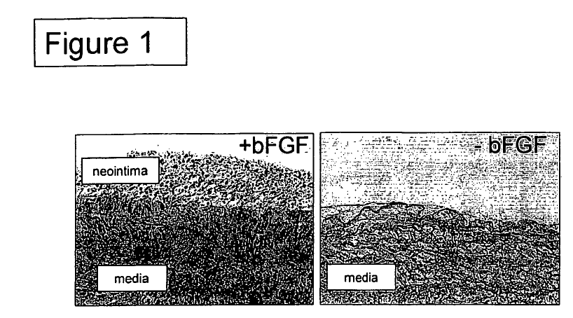

As can be seen in FIG. 1, in the pig aorta organ cultures, intimal hyperplasia can be induced by addition of basic fibroblast growth factor, 10 ng/ml, resulting a prominent neointima after 4-5 weeks in culture.

The same experiments were performed with segments of human aorta in organ cultures. Again, in human aortic organ cultures a clear induction of neointima was observed after 4-5 weeks in culture in the presence of 10 ng/ml bFGF.

### EXAMPLE 2

Vascular Prosthetic Material Coating and bFGF Release

Under sterile conditions, the standard woven vascular prosthetic material, Dacron® (Dupont), porosity 60 μm, was impregnated with collagen according the following previously described procedure. Type I insoluble collagen (1 g) derived from Bovine Achilles Tendon (Sigma, St Louis, Mo., USA) was swollen overnight in 0.52 M acetic acid solution (50 ml) at 4° C.

The mixture was dispersed with 50 g of crushed ice for 4 minutes in a blender and thereafter homogenized for 30 minutes at 4° C. using an Ultra-Turrax T25 (IKA Labortechnik, Staufen, Germany). The resulting slurry was filtered through a series of filters (Cellector screen, Bellco, Feltham, England), with a pore size decreasing from 140 μm to 10 μm, mounted in 47 mm diameter Swinnex disc filter holders (Millipore, Etten-Leur, The Netherlands).

After de-aeration at a pressure of 0.06 mBar, the resulting suspension was cast as a film on small pieces of Dacron® in sterile petri-dishes and dried at room temperature in a sterile flow cabinet. The Dacron®, pieces impregnated with collagen, were cross-linked using 0.731 g N-(-3-dimethylaminopropyl)-N′-ethylcarbodiimide hydrochloride (EDC) and 0.415 gN-hydroxysuccinimide (NHS). The crosslinking was carried out in 215 ml buffer of 2-morpholinoethane sulfonic acid (MES) buffer, 0.05 M, pH adjusted to 5.4 using 10 M NaOH, to minimize hydrolysis of EDC.

After 4 hours, the crosslinking was completed and stopped by washing the impregnated Dacron® with 0.1 M Na₂HPO₄ solution for 2 hours. This treatment resulted in hydrolysis of both activated carboxylic acid groups and remaining EDC. After repeated washing with sterile demineralised water (four times 30 minutes) the Dacron®, impregnated with cross-linked collagen was ready for heparin immobilization.

Heparin sodium salt (Bufa Chemie, Castricum, The Netherlands) was used. The carboxylic acid groups of heparin (Hep-COOH) were activated by adding EDC and NHS to a 2% solution of heparin in 0.05 M MES buffer (pH 5.6), at a molar ratio of EDC:NHS:Hep-COOH of 0.4:0.24:1.0. After 2 hours incubation the Dacron®/collagen/heparin was washed with 0.1 M Na₂HPO₄ for two hours, 4 M NaCl for four hours and distilled water for three times 24 hours. (Wissink M J, Beernink R, Scharenborg N M, Poot A A, Engbers G H, Beugeling T et al. Endothelial cell seeding of (heparinized) collagen matrices: effects of bFGF pre-loading on proliferation (after low density seeding) and pro-coagulant factors. J Control Release 2000; 67(2-3):141-155.; Wissink M J, Beernink R, Pieper J S, Poot A A, Engbers G H, Beugeling T et al. Immobilization of heparin to EDC/NHS-crosslinked collagen. Characterization and in vitro evaluation. Biomaterials 2001; 22(2):151-163.).

The binding of bFGF was carried out after incubating the Dacron®/collagen/heparin in phosphate buffered saline (PBS). After blotting dry, the films were incubated with 320-500 ng bFGF in PBS containing 1 mg/ml BSA for 90 minutes at room temperature.

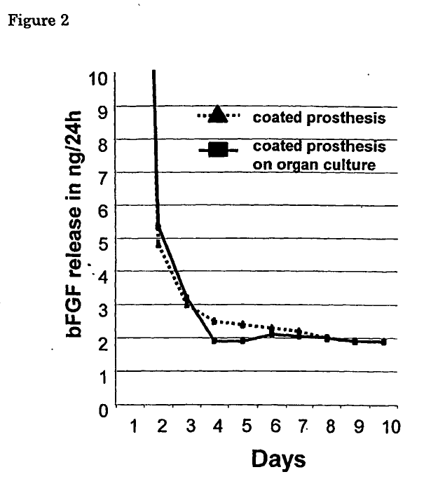

The release of bFGF was tested every 24 h in a sample obtained from the culture media in which the Dacron®/collagen/heparin/bFGF segments (1 cm diameter) were placed. After taken the sample every 24 hours, the medium was replaced. The concentration of bFGF in the release samples was determined using an ELISA for bFGF. The first 3 days a washout effect was detected followed by a stable release of bFGF of approximately 2 ng/24 h for 28 days (see FIG. 2 for the first 10 days). No major differences in long-term release could be observed after incubation with either 300 or 500 ng bFGF (not shown). The release of bFGF from the Dacron®/collagen/heparin/bFGF segments was also determined in the presence of organ cultures. Incubation of the Dacron® provided with bFGF/collagen/heparin resulted in a similar release of bFGF into the medium (FIG. 2). Binding of bFGF on the-collagen layer without heparin resulted in a quick release of bFGF in 4-5 days, after which no more release of bFGF could be detected.

### EXAMPLE 3

Quantification of Ingrowth of Neointima in Prosthesis

Experiments for inducing ingrowth of neointima in the vascular prosthetic material were performed as follows. Aortic tissue samples were obtained as previously described and directly attached to samples of one square cm of impregnated Dacron®. Experiments were performed with (a) unimpregnated Dacron® (control experiments), (b) collagen/heparin impregnated Dacron® (control experiments) and (c) collagen/heparin with growth factor impregnated Dacron®. Dacron® samples were attached to the aortic samples by either needles or paperclips. Subsequently, the samples were cultured for up to 35 days.

At different time points, 7, 14, 21, 28 and 35 days, the organ cultures with the prosthetic material were histologically evaluated using formaldehyde (4%) fixated, paraffin embedded tissue sections. After histochemical staining of section of the cultured vessel wall segments, the intimal hyperplasia was evaluated by computer assisted image analysis (Leica Imaging Systems, Cambridge, England). To asses the ingrowth into the Dacron® the number of cell layers was counted as well as the number of cells infiltrating in the Dacron®.

Ingrowth of Neointima in Prosthesis

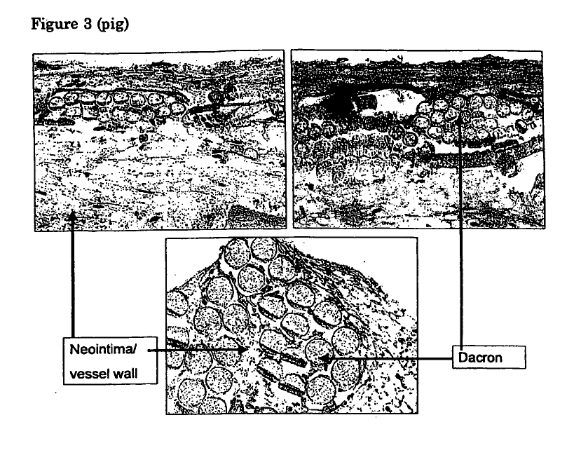

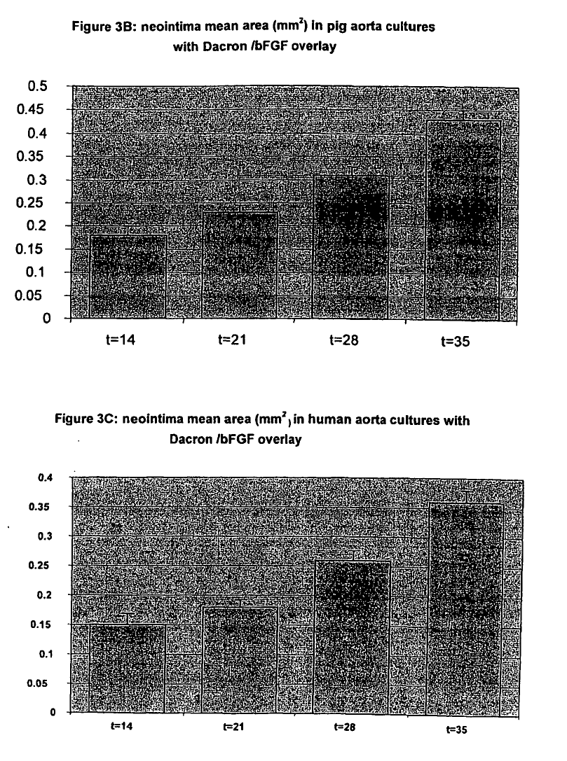

When organ cultures were grown with an overlay of the bFGF impregnated Dacron®, cells from the neointima start to migrate through the collagen impregnated prosthesis material, eventually resulting in a neointima on top of the prosthesis material (FIG. 3A). The cells migrated were shown to be smooth muscle cells that were α-SM actin positive by immunohistochemistry (data not shown). The induction of the neointima formation occurred only in porcine aorta organ cultures covered with prosthesis material provided with bFGF. Neointima formation was absent in porcine aorta organ cultures with pure Dacron®, Dacron® with collagen/heparin as well as in the control organ cultures without prosthesis material.

The ingrowth of neointima through the Dacron® was measured and quantified. The ingrowth of the neointima progressed in time with a mean neointima area per cross section of the cultured segments of 0.43±0.11 mm² after 5 weeks in culture (FIG. 3B).

Experiments using human aorta organ cultures showed comparable results. The human aortic organ culture were capable of formation of neointima, induced by the bFGF-collagen impregnated Dacron®. The cells of the neointima in the human aortic wall were capable of growing through the prosthetic material (FIG. 3C).

Neointima formation and cellular invasion through the prosthesis material could only be observed when the cultures were overlaid with bFGF impregnated Dacron® prosthesis material.

The neointimal ingrowth into the prosthesis material provided with collagen, heparin and bFGF resulted in a fixation of the prosthesis material to the vessel wall segments, both of porcine and human origin, in this in vitro model system.

### EXAMPLE 4

The fixation of the Dacron® prosthesis material provided with collagen and bFGF was exemplified using handmade stents prostheses of 2 cm in length that are positioned into the aorta of pigs.

For this, the diameter aorta of the pigs in the study first was measured using angiography. The custom-made stents were made to fit to the size of the aorta and prepared according the required conditions, i.e. non-coated, non-impregnated Dacron®; impregnated Dacron® without bFGF and impregnated Dacron® with bFGF. Provision of collagen, heparin and impregnation with bFGF was done as described above in example 2. The binding of bFGF was carried out after incubating the Dacron®/collagen/heparin in PBS. After blotting dry, the films were incubated with 320 or 500 ng bFGF in PBS containing 1 mg/ml BSA for 90 minutes at room temperature.

The stents were placed through a sheath (20 French pusher, Cook Denmark) under angiographic control and were left in the aorta for 2 months. After two months the pigs were sacrificed and the aortas with the stents were evaluated macroscopically, histologically and by scanning electron microscopy.

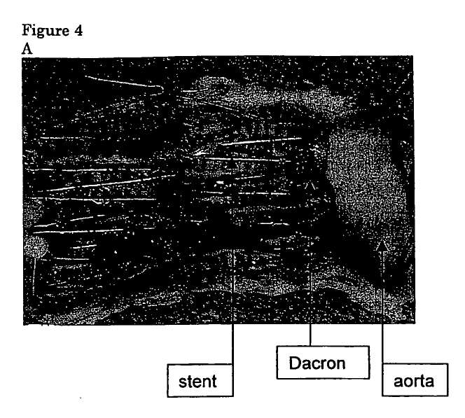

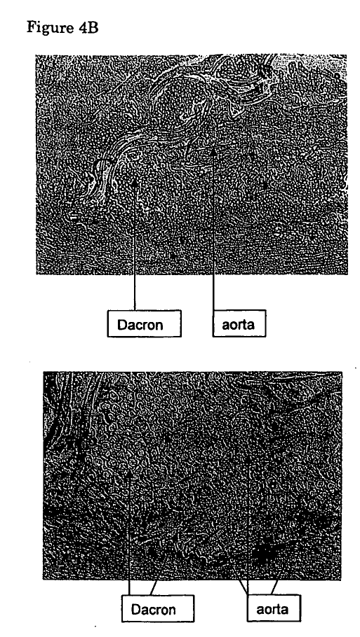

Macroscopic analysis revealed that a firm adhesion of the prosthesis material to the aorta wall occurs when bFGF impregnated Dacron® prosthesis material were used, indicating that the graft material had healed to the vessel wall. In none of the other conditions adhesion, and thus healing, occurred. As can be seen in FIG. 4A, two months after initial placement of the Dacron® prosthesis, it is fixed to the vessel wall of the aorta in such a way that it does not detach, not even after cutting the stent material.

Histological analysis demonstrated that at the moment of sacrifice, two months after initial placement, the bFGF containing impregnated Dacron® graft material was fully incorporated into the vessel wall (FIG. 4B), whereas the controls were not incorporated.

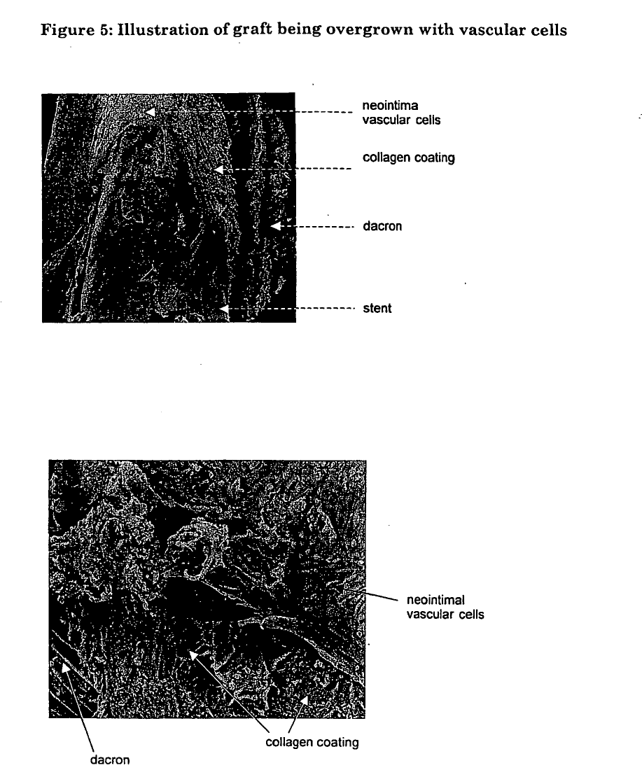

FIG. 5 shows two scanning electron micrographs, illustrating how the graft according to this example was overgrown with neointimal vascular cells.

1. A vascular prosthesis, comprising:
a synthetic basic structure, which structure comprises voids for holding vascular cells; and
an active substance that has the capacity to stimulate the ingrowth of vascular cells into the basic structure operatively associated with the basic structure.

2. The vascular prosthesis of claim 1, wherein the active substance is one of an inducer and a stimulator for one of the production of connective tissue, the deposition of connective tissue, smooth muscle cell proliferation, smooth muscle cell migration, intimal hyperplasia and neointima formation.

3. The vascular prosthesis of claim 1 wherein the active substance is chosen from the group consisting of:
(a) basic fibroblast growth factors;
(b) acidic fibroblast growth factors;
(c) platelet derived growth factors;
(d) transform growth factors beta;
(e) another growth factor which is at least one of an inducer and a stimulator for one of the production of connective tissue, the deposition of connective tissue, smooth muscle cell proliferation, smooth muscle cell migration, intimal hyperplasia and neointima formation; and
(e) a combination of said growth factors.

4. The vascular prosthesis of claim 1, wherein the active substance, is capable of stimulating fibrosis.

5. The vascular prosthesis of claim 1, wherein the active substance is applied to the basic structure as one of a coating, lining and impregnation;

6. The vascular prosthesis of claim 5, wherein the coating, lining or impregnation comprises a compound selected from the group consisting of collagen, heparin, gelatin, fibrin, elastin, glycosaminoglycans, fibronectin, laminin, proteoglycans, albumin, globulin, polyactide, polyglycolide, polygluconate, polydioxanon and a combination of two or more of these compounds.

7. The vascular prosthesis of claim 1, wherein the synthetic basic structure comprises a material selected from the group consisting of polycondensate of ethylene glycol and terephtalic acid polytetrafluorethylene, polyamide, polyester, metal, metal alloy, polycarbonate-urethane, silicon, polyolefine and a combination of two or more of these materials.

8. The vascular prosthesis of claim 1, wherein the active substance is released under physiological conditions in a controlled manner.

9. The vascular prosthesis of claim 1, wherein a portion of the voids in the synthetic basic structure are pores.

10. The vascular prosthesis of claim 1, wherein the mean void diameter of the voids in the synthetic basic structure is approximately 5-150 μm.

11. The vascular prosthesis of claim 1, wherein the vascular prosthesis is one of vascular graft and an artificial heart valve.

12. The vascular prosthesis of claim 1, wherein the vascular prosthesis is a vascular graft, the vascular prosthesis further comprising a stent and a sheath, wherein the stent is positioned in a central passage way of the vascular graft, wherein the stent and the vascular graft are resiliently compressed to allow expansion in the radial direction and wherein the outer surface of the compressed graft is surrounded by a cylindrical sheath, wherein the vascular prosthesis has a diameter and length suitable for catheter-based delivery to a vascular segment to be treated.

13. A method of preparing a vascular prosthesis, comprising:
providing a basic structure comprising voids; and
applying an active substance to the basic structure capable of locally inducing the ingrowth of vascular cells into the basic structure.

14. The method of claim 13, wherein the active substance is applied by one of coating, impregnating and lining the basic structure.

15. The method of claim 14, wherein the basic structure is first provided with collagen, subsequently with heparin and thereafter with the active substance.

16. The method of claim 13, wherein the active substance is a growth factor.

17. A method of using a growth factor, the method comprising:
manufacturing a medicament for stimulating the ingrowth of vascular cells into an endovascular prosthesis, wherein the prosthesis is formed of a synthetic basic structure, which synthetic basic structure comprises voids for holding vascular cells; and
adding growth factor to the medicament.

18. The method of claim 17, wherein the administering-unit of the medicament is the endovascular prosthesis.

19. The method of claim 17, wherein the medicament is for treatment of an aneurism.

20. The method of claim 19, wherein the medicament is for treatment of an aneurism by a catheter-delivery based minimally invasive endovascular technique.

21. A method for the treatment of a vascular disorder, said method comprising the placement of the endovascular prosthesis of claim 1 in the vascular system of a patient.

22. The method of claim 21, wherein the prosthesis is placed into the vascular system by a catheter-delivery based minimally invasive endovascular technique.

23. The vascular prosthesis of claim 5, wherein the coating, lining or impregnation is partially biodegradable.

24. The vascular prosthesis of claim 7, wherein the synthetic basic structure comprises a material selected from the group consisting of Dacron®, Telfon®, nylon and polyethene and a combination of two or more of these materials.

25. The method of claim 16, wherein the growth factor is a fibroblast growth factor.

26. The method of claim 17 wherein the growth factor is a fibroblast growth factor.
